(19) United States
(12) Patent Application Publication
(10) Pub. No.: US 2006/0134625 A1
(43) Pub. Date: Jun. 22, 2006

(54) Method for determining susceptibility to schizophrenia

(76) Inventors: Michel Maziade, Quebec (CA); Chantal Merette, Quebec (CA); Marc-Andre Roy, Quebec (CA)

Correspondence Address: OGILVY RENAULT LLP, 1981 MCGILL COLLEGE AVENUE, SUITE 1600, MONTREAL, QC, H3A2Y3, CA

(21) Appl. No.: 10/546,046

(22) PCT Filed: Feb. 19, 2004
(86) PCT No.: PCT/CA04/00229

§ 371 Date: Feb. 22, 2006

(30) Foreign Application Priority Data
Feb. 19, 2003 (US) 60447704

(51) Int. Cl. C12Q 1/68

(52) U.S. Cl. 435/6

(57) ABSTRACT

The present invention relates to methods for identifying the susceptibility or predisposition of an individual to schizophrenia SZ) or to clusters of symptoms associated thereof. Particularly, the tests can be performed before or after the disorders appear. More particularly, the present invention relates to the determination of epistatic effect of at least two genotype related loci. In addition, the invention provides tests for the classification of different subtypes predicting severity of illness of patients affected by or predisposed to SZ or associated clusters of symptoms.

### TECHNICAL FIELD

The present invention relates to the diagnosis of the genetic susceptibility of a patient to schizophrenia (SZ) and to several symptoms associated therewith. More particularly, the invention relates to combinations of genotype markers and their inter-relation allowing more precisely than the methods existing in the art for determining whether an individual has a predisposition for developing SZ. The present invention relates also to the diagnosis of this disease after it appears and to the prognosis of its severity.

### BACKGROUND ART

On one particular side, although the cause of SZ is still relatively unknown, family and adoption studies suggest that SZ has a significant genetic component. As in most common diseases, the inheritance pattern is complex, and the penetrance is low. Unfortunately, there is no reliable biological marker for the disease. The many ways that SZ manifests itself could be explained by locus heterogeneity, which also may have hampered progress in the search for SZ genes. Given that neurotransmitter pathways are probably abnormal in SZ, variants of genes encoding receptor subunits and transporters have been assessed for association to SZ in many populations.

Although there have been reports of weak association with variations of certain genes, no significant findings have been reproduced in independent cohorts. The associations reported so far, if real, represent only minor genetic contributions. Genetic linkage has been studied and linkage maps have been developed for a wide variety of species, including plant species. Localization of genes of interest can be accomplished through linkage analysis with mapped markers as described by Patterson, E. B. (1982) “The mapping of genes by the use of chromosomal aberrations and multiple marker stocks”, pp. 85-88, In: Maize for Biological Research (W. F. Sheridan, ed.) University Press, University of North Dakota, incorporated herein by reference.

Different genome-wide linkage scans have been reported for SZ. There is modest evidence for linkage with several loci, including chromosome 1q, 2, 6, 8p, 13q, and 22q. Chromosome locus 8p (SCZD6 (MIM 603013)) shows suggestive linkage to SZ in several different populations.

U.S. Pat. No. 6,225,057 discloses a method for identifying a person at risk of developing different anxiety disorders by performing a correlation with genotype markers in a limited region of the chromosome 15. This study is however defined as using only one marker at time.

U.S. Pat. No. 6,136,532 discloses a method for predicting a patient's likelihood for developing bipolar disorders. The method, while being based on the analysis of genomic markers, it is limited to a restricted region of the chromosome 18. The concept of using genomic markers associated with physical traits to track and recover this traits in segregating populations is now known to the art. While nucleic acid (RFLP) markers have been used to locate and manipulate traits determined by single genes, they have not been successfully used to locate and manipulate, when needed, traits determined by more than one gene.

The identification of a gene of susceptibility to SZ (neuregulin) located at 8p seems likely possible. However, no evidence for an interaction between the neuregulin gene and genes or loci located at different chromosomal areas is yet known.

A research group has recently published (Straub et al. 2002) results for the identification of a gene of susceptibility to SZ (dysbindin) located at 6p22.3. However, no evidence for an interaction between the dysbindin gene and genes or loci located at different chromosomal areas was published.

A weakness in the use of molecular markers for tracking and hereditable traits is the fact that crosses-over occurring in progeny predictably can separate the trait of interest from the linked marker used to track it in a certain percentage of individuals.

Another weakness of prior methods for tracking traits or heritable disorders using molecular markers is the fact that a particular linked marker allele may not invariably correlate with the presence of the phenotype being studied. Many phenotypes are developmentally expressed, and unless the populations are scored at multiple times during their life cycles, important associated marker alleles can fail to be identified.

It would be highly desirable to be provided with a new method for an improved identification of the susceptibility of an individual to heritable disorder. More particularly, epistasis and combination of genomic markers located on different chromosomes would be highly more efficient in this goal.

### DISCLOSURE OF THE INVENTION

One object of the present invention is to provide tests to identify, preferably before the illness appears, susceptibility or vulnerability of an individual to SZ or to clusters of symptoms associated with this neuropsychiatric disorder.

Another object of the present invention is to provide tests for diagnosis and/or prognosis of SZ after the disorder appear or for diagnosis of clusters of disease symptoms.

Also one object of the present invention is to provide tests for the classification of different sub-groups of patients affected by or predisposed to SZ or spectrum of disorders. Several kinds of theranostic tests are additionally provided.

The present invention relates to an epistatic effect made by combining two susceptibility loci as for example 6p22.3 (LDB; http://cedar.genetics.soton.ac.uk/pub/chrom6/gmap) and 18q21.1 (LDB; http://cedar.genetics. soton.ac.uk/pub/chrom18/gmap).

In accordance with the present invention there is provided a method for developing new treatments derived from the defective genes located at least at loci: 6p22.3 and 18q21.1.

Another aspect of the present invention is to provide a new method for disease phenotype definitions thanks to dimensional and syndromal phenotype characterization.

One object of the present invention is to provide a method for determining the susceptibility, before the illness appears, of an individual to develop at least SZ or associated clusters of symptoms thereof comprising characterizing in a tested individual a genotype markers combination of at least two genotype markers substantially equivalent to genotype markers in a patient diagnosed for SZ or associated clusters of symptoms thereof, the genotype markers being selected from the group consisting of markers located in genotype region 6p22.3 and 18q21.1, wherein the characterization of the genotype markers combination of the tested individual is representative of the susceptibility of the individual to develop at least one of SZ or associated clusters of symptoms thereof.

Another object of the invention is to provide a method for diagnosing illness or predicting severity of illness of at least one of SZ or associated clusters of symptoms thereof of a patient comprising characterizing in a tested patient genotype marker combination of at least two genotype markers substantially equivalent to genotype markers in a patient diagnosed for SZ or associated clusters of symptoms thereof, the genotype markers being selected from the group consisting of markers located in genotype region 6p22.3 and 18q21.1, wherein the characterization of the genotype markers combination of the tested patient is representative of the presence of illness or the severity of illness of at least one of SZ or associated clusters of symptoms thereof of the patient.

In accordance with the present invention it is provided a method for determining susceptibility of an individual to develop at least one of SZ or associated clusters of symptoms thereof before the illness appears comprising;
a) characterizing in an individual a genotype markers combination of at least one genotype markers located in genotype region 6p22.3 and at least one genotype marker located in genotype region 18q21.1; and
b) comparing genotype markers of the individual of step a) with genotype markers of a patient diagnosed for SZ or associated clusters of symptoms thereof located in genotype regions 6p22.3 and 18q21.1, wherein detecting presence of genotype markers of the patient diagnosed for SZ or associated clusters of symptoms thereof in genotype regions 6p22.3 and 18q21.1 of the individual is representative of the susceptibility of to develop at least one of SZ or associated clusters of symptoms thereof.

It will be recognized by those skilled in the art that the level of correlation of the genotype markers of a tested individual with genotype markers of a patient diagnosed for SZ or associated clusters of symptoms thereof is representative of the level of the predisposition of a tested patient to develop SZ or associated clusters of symptoms thereof.

The genotype markers combination is generally an epistatic combination or markers. The effect of the combination is mostly synergistic, in the sens that each gene is necessary but not sufficient when taken individually to develop SZ or associated clusters of symptoms. This may result in a prognostic, a diagnostic, or characterization of responsiveness with efficiency of preferably 60%. Depending of the cluster and markers targeted, the efficiency may vary of between about 20 to 80% while remaining far more efficient than other methods available in the art.

Also, the genotype markers pattern of a tested individual may be of about 50 to 100% corresponding to or shared with genotype markers pattern of an affected patient diagnosed for SZ or associated clusters of symptoms thereof. The affected patient can be clinically or genetically diagnosed or a combination of both method.

It can also be admitted in the art that the SZ can be a schizophreniform disorder, an achizotypal personality disorder, psychosis or a schizoaffective disorder.

The characterization of the genotype markers is preferably carried out by determining the DNA sequence of these genotype markers.

Preferably, the epistatic genotype markers combination, named according to the National Center for Biotechnology Information (NCBI; Build 34 version 2; http://www.ncbi.nlm.nih.gov/) includes combination of at least one marker between loci D18S65 and D18S64 on chromosome 18 (Genetic Location Database (LDB) version Mar. 26 2001; http://cedar.genetics.soton.ac.uk/pub/chrom18/map.html) and at least one marker located between loci D6S1267 and D6S89 on chromosome 6 (LDB version Nov. 9 1999; http://cedar.genetics.soton.ac.uk/pub/chrom6/map.html)

The diagnosis or prediction can be performed before or after symptoms of at least one of SZ or associated clusters of symptoms thereof occurs.

For the purpose of the present invention the following terms are

The term “genetic marker” as used herein is intended to mean a locus whose alleles are readily detectable. It may or may not be part of an expressed gene. Genetic marker includes characteristics with a ready classification into different phenotypes, a simple mode of inheritance and different frequencies in different population. This may be defined as a locus, or a gene of known function and known location on the chromosome, or any distinct phenotype, determined by a single gene or mutant allele, that can be used in experimental genetics for such purposes as estimating the linkage distance between two loci in recombination analysis. This can be alternatively defined as genetic polymorphism with a simple mode of inheritance occurring with different frequencies in different populations, and therefore useful in family studies, studies of the distribution of genes in populations, and linkage analysis.

The term “locus” as used herein is intended to mean the position on a chromosome at which the gene for a particular trait resides; locus may be occupied by any one of the alleles for the genes.

The expression “LOD score” as used herein is intended to mean the measure of genetic linkage, defined as the log10 ratio of the probability that the data would have arisen if the loci are linked to the probability that the data could have arisen from unlinked loci. The conventional threshold for declaring linkage is a LOD score of 3.0, that is, a 1000:1 ratio.

The terms “epistasis” or “or coactivity” or “coactive” as used herein is intended to mean a situation in which the interaction of two genes is needed to produce a phenotypic effect in a given individual. Epistasis or coactivity occurs when the combined effect of two genes on a phenotype exceeds the sum of their separate effect. When two genes, for example genes A and B, act in epistasis, each gene is necessary but not sufficient to fully explain the related phenotype. Genes A and B both need to be present in a particular subject or patient for the disease to be developed. Epistasis may explain the difficulty in obtaining unambiguous linkage evidence for psychiatric disorders despite their high level of heritability. Indeed, genes involved in epistasis may only be detectable when considered within a model allowing for an interplay among genes while most current methods for detecting genes do not allow or such interplay and rather treat one gene at a time. Epistasis can alternatively be defined as being a synergetic or coactive effect between two or more genes of parts thereof.

### BRIEF DESCRIPTION OF DRAWINGS

FIG. 1 illustrates two-point mod score curves for each diagnostic hierarchy (SZ, BP, CL=common or shared locus) providing the level of affection (either narrow or broad) for which the highest mod score was obtained for a given chromosome;

FIG. 2 illustrates haplotypes showing an epistatic effect between chromosomes 6 and 18 to cause SZ.

FIG. 3 illustrates examples of enrolled pedigrees.

### MODE OF CARRYING OUT THE INVENTION

The present invention now will be described more fully hereinafter with reference to the accompanying drawings, in which preferred embodiments of the invention are shown. This invention, may, however, be embodied in many different forms and should not be constructed as limited to the embodiments set forth herein; rather, these embodiments are provided so that this disclosure will be thorough and complete, and will fully convey the scope of the invention to those skilled in the art.

In accordance with the present invention, there is provided a method for diagnosing with accuracy improved, in regard to other methods known in the art, patients susceptible of developing phenotypic manifestations of SZ or related disorders with all their different characteristics and traits. This invention could also allow to develop therapeutic tools for treating SZ or related disorders.

Particularly, one embodiment of the present invention is to predict the susceptibility of a patient to SZ by using combination of genotype markers. More particularly, on chromosome 18 (Table 1) the markers targeted through the method of invention are found between the loci D18S65 and D18S64, and on chromosome 6 (Table 2), between the loci D6S1267 and D6S89. The predictive correlation of the susceptibility to SZ is highly improved as compared to the current state of the art. The method invention also finds a synergistic effect of the combination which gives much better results than just the addition of the two loci.

TABLE 1
Two-locus lod scores on chromosome 18 testing
for epistasis with chromosome 6.

| | Position^{a} | Two-locus lod score |
|---|---|---|
| Marker | (cM) | Epistasis |
| D18S65 | 45.27 | 4.04 |
| D18S1145 | 46.55 | |
| D18S467 | 47.59 | |
| D18S455 | 47.85 | |
| D18S472 | 48.37 | 8.25 |
| D18S474 | 49.66 | 3.33 |
| D18S851 | 50.61 | 7.64 |
| D18S69 | 53.13 | |
| D18S858 | 54.92 | |
| D18S41 | 54.8 | 3.98 |
| D18S64 | 60.08 | 2.37 |

^{a}according to LDB genetic map (version Mar26 2001)

TABLE 2
Two-locus lod scores on chromosome 6 testing
for epistasis with chromosome 18.

| | Position^{a} | Two-locus lod score |
|---|---|---|
| Marker | (cM) | Epistasis |
| D6S1267 | 15.75 | 5.15 |
| D6S1605 | 15.94 | |
| D6S334 | 16.00 | 8.25 |
| D6S274 | 16.07 | |
| AF189YE3 | 16.36 | |
| D6S1676 | 16.61 | |
| D6S469 | 16.82 | |
| D6S285 | 18.84 | |
| D6S1959 | 23.28 | 7.64 |
| D6s1050 | 26.09 | |
| D6S259 | 27.78 | 6.79 |
| D6S2439 | 28.79 | |
| D6S89 | 29.92 | 3.54 |

^{a}according to LDB genetic map (version Nov9 1999)

The present invention will be more readily understood by referring to the following examples which are given to illustrate the invention rather than to limit its scope.

### EXAMPLE I

### Linkage results in sample 1.

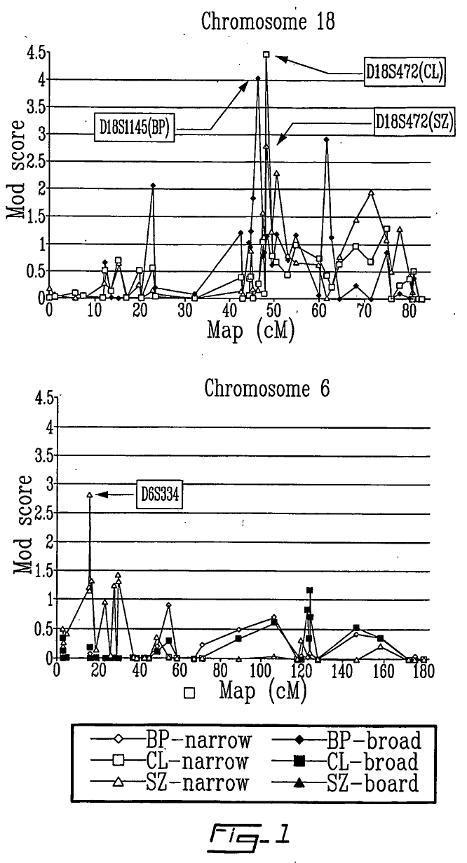

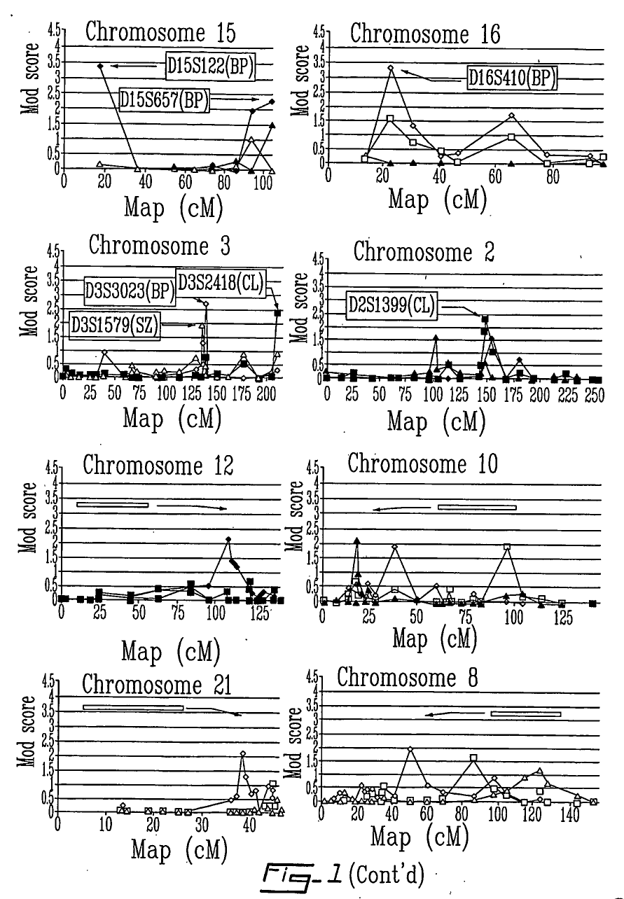

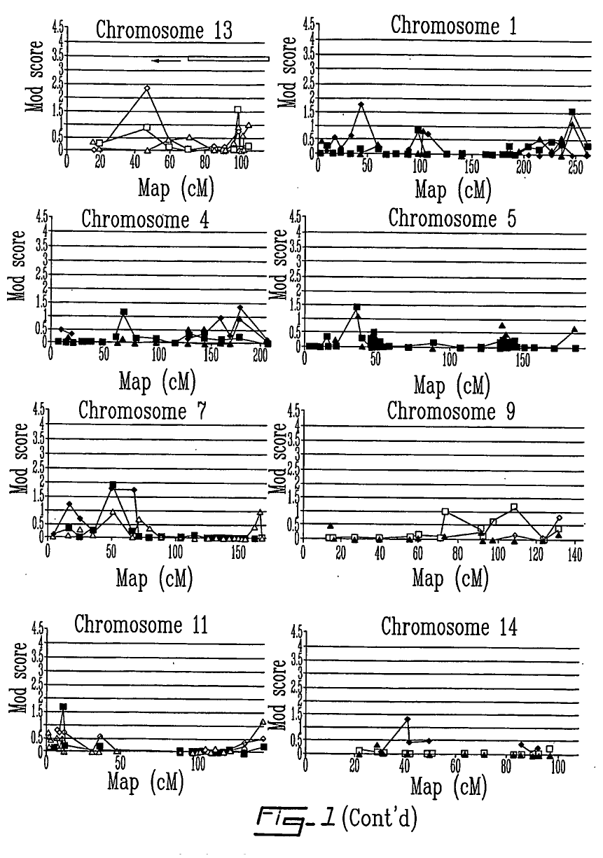

Genome scan in sample 1 was completed, comprising 480 markers (i.e., 350 markers in a 10 cM resolution map plus 130 additional markers to follow up positive results). FIG. 1 now shows the results of the full genome scan. This genome scan yielded several linkage signals that were classified as being either significant, suggestive or confirmatory according to conservative thresholds derived by increasing the criteria by 0.70 to correct for multiple testing. Hence, the adjusted lod score criteria were 4.0 for a genome wide significant linkage, 2.6 for a suggestive and 1.9 for a confirmatory linkage (i.e. a region where significant linkage was shown such as in 6p22.3 for SZ). Table 3 lists the 7 results where a lod score met at least the suggestive level of significance. Among these 7 loci, two met the criterion for “genome wide” significance: a lod score of 4.46 was found at D18S472 (18q21.1) with the common locus (CL) phenotype, and a lod score of 4.03 was found at D18S1145, a locus two centiMorgan (cM) away from D18S472, using the BPbroad phenotype. The 18q results are congruent with linkage evidence for BP in that area. Interestingly, a L & K suggestive linkage at D18S851 (Z=3.87) for the SZ phenotype was obtained, which suggested a region of linkage shared by SZ and BP. Moreover, at 6p22.3 (D6S334), a lod score of 2.8 under homogeneity and 3.47 under heterogeneity with SZ was obtained, meeting the criterion for confirmation of a significant linkage.

TABLE 3
Two-point and three-point mod score meeting the criteria for significant (Z ≧ 4.0),
confirmatory linkage (Z ≧ 2.6, with previous significant linkage), suggestive
linkage (Z ≧ 2.6) or other linkage signal (Z ≧ 1.9).

| | | Two-point | | | | | | |
|---|---|---|---|---|---|---|---|---|
| | | | Map | Mode of | Homogeneity | Heterogeneity | | |
| Region | Phenotype^{a} | Marker | (cM) | inheritance^{b} | Zmax (θ) | Zhet | α | θ |

Significant linkage (Zmax ≧ 4.0)

| | | | | | | | | |
|---|---|---|---|---|---|---|---|---|
| 18q21.1 | CL′nar | D18S472 | 48.4 | R-AO | 4.46 (0.02) | 4.46 | 1.00 | 0.02 |
| 18q12.3 | BPbro | D18S1145 | 46.6 | R-AU | 4.03 (0.15) | 4.03 | 1.00 | 0.15 |

Confirmatory linkage (Zmax ≧ 2.6 and significant finding in a previous study)

| | | | | | | | | |
|---|---|---|---|---|---|---|---|---|
| 6p22.3 | SZnar | D6S334 | 16.0 | D-AO | 2.82 (0.10) | 3.47 | 0.66 | 0.00 |

Suggestive linkage (Zmax ≧ 2.6)

| | | | | | | | | |
|---|---|---|---|---|---|---|---|---|
| 16p12.3 | BPnar | D16S410 | 22.6 | D-AO | 3.30 (0.00) | 3.30 | 1.00 | 0.00 |
| 18q21.1 | SZnar | D18S851 | 50.6 | D-AU | 3.87 (0.10) | 3.87 | 1.00 | 0.10 |
| 15q11.1 | BPbro | D15S122 | 17.6 | D-AO | 3.35 (0.00) | 3.35 | 1.00 | 0.00 |
| 3q21.2 | BPnar | D3S3023 | 138.9 | D-AO | 2.68 (0.05) | 2.68 | 1.00 | 0.05 |

Other linkage signal (Zmax ≧ 1.9)

| | | | | | | | | |
|---|---|---|---|---|---|---|---|---|
| 2q12.3 | CLnar | D2S121 | 114.9 | R-AO | 2.18 (0.10) | 2.18 | 1.00 | 0.10 |
| 2q22.1 | CLbro | D2S1399 | 148.4 | D-AU | 2.27 (0.25) | 2.27 | 1.00 | 0.25 |
| 3q21.1 | SZnar | D3S1579 | 135.4 | R-AO | 1.91 (0.05) | 1.96 | 0.72 | 0.00 |
| 3q29 | CLbro | D3S2418 | 210.9 | D-AU | 2.37 (0.25) | 2.37 | 0.95 | 0.25 |
| 8p11.1 | BPnar | D8S1110 | 50.2 | D-AU | 1.90 (0.15) | 2.18 | 0.40 | 0.00 |
| 10p13 | SZbro | D10S245 | 18.1 | D-AU | 2.06 (0.15) | 2.41 | 0.68 | 0.10 |
| | BPnar | — | — | — | — | — | — | — |
| 11p15.4 | BPbro | — | — | — | — | — | — | — |
| | CLbro | — | — | — | — | — | — | — |
| 12q23.2 | BPbro | IGF1 | 107.2 | D-AO | 2.06 (0.10) | 2.06 | 1.00 | 0.10 |
| 13q14.11 | BPnar | D13S325 | 46.6 | D-AO | 2.27 (0.05) | 2.27 | 1.00 | 0.05 |
| 15q26.3 | BPbro | D15S657 | 104.6 | R-AO | 2.31 (0.05) | 2.31 | 1.00 | 0.05 |
| 16q21 | BPbro | D16S3253 | 65.0 | D-AO | 2.19 (0.01) | 2.19 | 1.00 | 0.01 |
| 21q22.13 | BPnar | D21S1893 | 38.6 | D-AU | 2.03 (0.15) | 2.03 | 1.00 | 0.15 |

| | | Three-point | | |
|---|---|---|---|---|
| | | | Mode of | |
| Region | Phenotype^{a} | Markers and θ's | inheritance^{b} | Zmax |

Significant linkage (Zmax ≧ 4.0)

| | | | | | | | | |
|---|---|---|---|---|---|---|---|---|
| 18q21.1 | CL′nar | D18S1145 | 0.14 | D18S472 | 0.10 | CL | R-AO | 3.34 |
| 18q12.3 | BPbro | BP | 0.20 | D18S1145 | 0.06 | D18S455 | R-AU | 4.10 |

Confirmatory linkage (Zmax ≧ 2.6 and significant finding in a previous study)

| | | | | | | | | |
|---|---|---|---|---|---|---|---|---|
| 6p22.3 | SZnar | SZ | 0.20 | D6S334 | 0.02 | D6S274 | D-AO | 2.00 |

Suggestive linkage (Zmax ≧ 2.6)

| | | | | | | | | |
|---|---|---|---|---|---|---|---|---|
| 16p12.3 | BPnar | D16S410 | 0.00 | BP | 0.13 | D16S403 | D-AO | 3.91 |
| 18q21.1 | SZnar | D18S455 | 0.07 | SZ | 0.02 | D18S472 | R-AO | 2.90 |
| 15q11.1 | BPbro | D15S122 | 0.00 | BP | 0.19 | D15S165 | R-AO | 2.41 |
| 3q21.2 | BPnar | BP | 0.10 | D3S3023 | 0.17 | D3S1764 | D-AO | 2.66 |

Other linkage signal (Zmax ≧ 1.9)

| | | | | | | | | |
|---|---|---|---|---|---|---|---|---|
| 2q12.3 | CLnar | — | — | — | — | — | — | — |
| 2q22.1 | CLbro | — | — | — | — | — | — | — |
| 3q21.1 | SZnar | — | — | — | — | — | — | — |
| 3q29 | CLbro | D3S1262 | 0.16 | D3S2418 | 0.30 | CL | D-AU | 2.03 |
| 8p11.1 | BPnar | — | — | — | — | — | — | — |
| 10p13 | SZbro | — | — | — | — | — | — | — |
| | BPnar | D10S2325 | 0.09 | BP | 0.00 | D10S674 | D-AO | 2.43 |
| 11p15.4 | BPbro | D11S1331 | 0.04 | D11S1999 | 0.20 | BP | D-AO | 2.14 |
| | CLbro | D11S1331 | 0.04 | D11S1999 | 0.30 | CL | D-AU | 1.93 |
| 12q23.2 | BPbro | — | — | — | — | — | — | — |
| 13q14.11 | BPnar | D13S325 | 0.00 | BP | 0.15 | D13S119 | D-AO | 2.20 |
| 15q26.3 | BPbro | — | — | — | — | — | — | — |
| 16q21 | BPbro | — | — | — | — | — | — | — |
| 21q22.13 | BPnar | — | — | — | — | — | — | — |

^{a}Phenotypes are schizophrenia narrow (SZnar) or broad (SZbro), bipolar narrow (BPnar) or broad (BPbro) and common locus narrow (CLnar) or broad (CLbro).
^{b}Modes of inheritance are either dominant (D) or recessive (R) and either affected-only (AO) or affected/unaffected (AU).
Note:
The “—” indicates that the corresponding two-point or three-point result did not meet the above criteria.

Evidence of an Epistatic Effect

Epistasis is said to occur when the combined effect of two genes on a phenotype exceeds the sum of their separate effect. Epistasis may explain the difficulty in obtaining unambiguous linkage evidence for psychiatric disorders despite their high level of heritability. Indeed, genes involved in epistasis may only be detectable or may have a stronger effect when considered within a model allowing for epistasis. Evidence for epistasis has recently emerged from the literature for various complex disorders such as asthma, sporadic breast cancer, systemic lupus erythematosus, type 2 diabetes.

It has been proposed a way to detect epistasis through a thorough investigation of correlations between family lod scores resulting from simple single-locus (SL) analyses: “Under epistasis SL lod scores tend to be positively correlated among pedigrees, while under independent action SL lod scores from high-density samples tend to be negatively correlated.” Once a positive correlation has been detected, the final test is a two-locus lod score calculated to obtain a global index of epistasis. In such two-locus analysis, the probability of linkage at two interacting loci is estimated simultaneously.

This three-step approach was used here to detect potential epistatic effects considering first the performing of single-locus analyses then, as an initial screening, a thorough investigation of correlation pattern between the significant, confirmed and suggestive loci identified through the genome scan of sample 1 that pertains to the same phenotype. Within each phenotype, a Bonferonni correction to the p-value used was applied to assess the significance of the correlation and obtained corrected p-values of 0.05 and 0.0125 for SZ and BP, respectively. After this correction, only a few correlations remained significant: For example, the family lod scores at D18S472 correlated with the family lod scores at D6S334 in the SZ families (r=0.54, p=0.01). A two-locus lod score was then calculated and weighted according to the evidence of the family's contribution to the lod score at D6S334, which provided a global assessment of both epistasis and heterogeneity. This yielded a weighted two-locus lod score of 8.25, meaning that the evidence of epistasis under heterogeneity between D6S334 and D18S472 for SZ is 100,000,000 times that of no linkage. This result is the first piece of evidence for a epistasis in psychiatric disorder research.

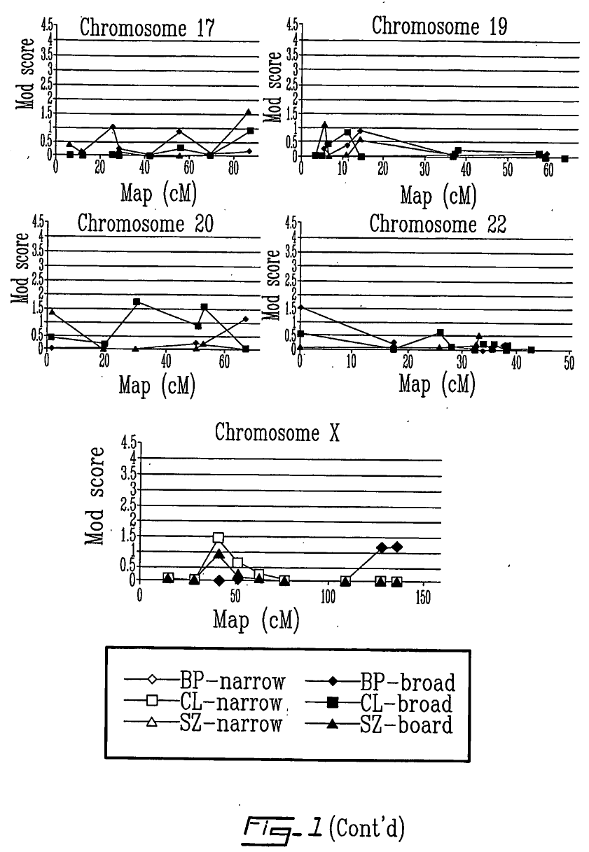

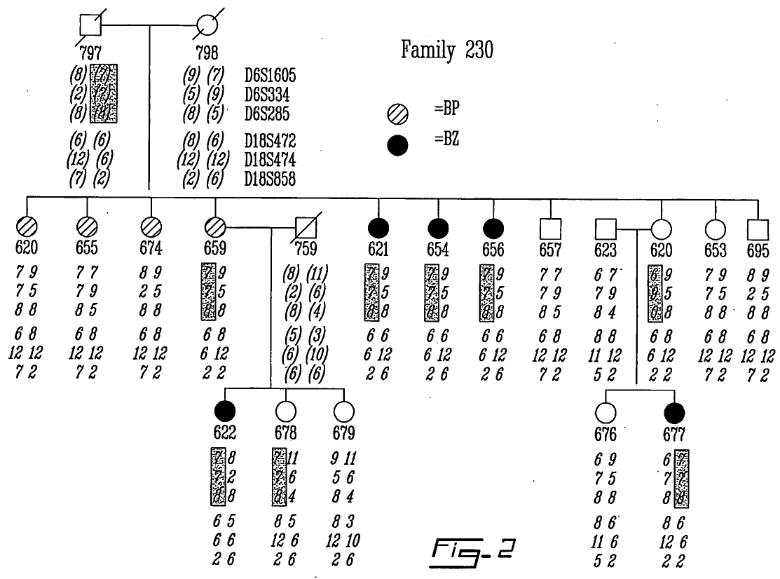

FIG. 2 illustrates one of the pedigrees that contributed to this epistasis finding. Haplotypes on chromosomes 6 and 18 were constructed for each subject within the family to validate the finding. One can indeed see that only the subjects affected by SZ, or those who transmitted SZ (subjects 659 and 690), shared both the haplotypes on 6 and 18. Subjects having only one of these two possible haplotypes did not express SZ.

### EXAMPLE II

### The Patient Population and its Characteristics

The population of Canada, especially Eastern Quebec, has unique assets for genetic studies based on 1) the geographic stability; 2) a universal health care system allowing for easier sampling and ascertainment; 3) an easy access to intact church baptism and marriage registers since the 17th century; 4) a large sibships in older generations. Hence, increasing sample size, as it was done, a major objective allowed to take full advantage of these unique characteristics.

The screening procedure of the field organization has been established since the early nineties. The pedigrees were ascertained through screening of the medical archives or through direct referral from clinicians from the clinical psychiatry departments in metropolitan Québec and surrounding regions of Eastern Québec The ascertainment procedure was approved by the medical directors and by the ethics committee.

Ascertainment was conducted in 3 regions: Beauce (pop.=100,000), Saguenay Lac St-Jean (140,000) and Iles-de-la-Madeleine (15,000). Due to the social pressures of an earlier time, large sibships made up of 7 to 15 persons were prevalent in Québec society. The entry criteria for a pedigree, based on “field” information, were: 1) At least three 1^{st} degree relatives affected by DSM-III-R SZ or BP; 2) At least four affected subjects in the whole pedigree (1^{st}, 2^{nd} or 3^{rd} degree); 3) As many unaffected subjects in the pedigree. The pedigree resources was not yet fully exploited in the population since 19 additional pedigrees meeting the entry criteria are awaiting enrollment depending on future needs.

Bilineality was assessed not to exclude families but to eventually incorporate this information in the context of a high probability of oligogenic transmission. To do so, spouses were systematically interviewed about themselves but also about their first and second-degree relatives and the co-parent was asked about the relatives of the married-in spouses.

Genealogical searches were based on systematic and official parish and government records for birth and marriage going back to the 17th century. These genealogies showed that none of the pedigrees were related up until the sixth generation. The field organization and the geographic stability of the population allow us to follow-up pedigrees and to regularly check for appearance of newly affected family members. In the past two years only, this follow-up procedure has provided 24 new incident cases in samples 1 and 2.

Present Sample

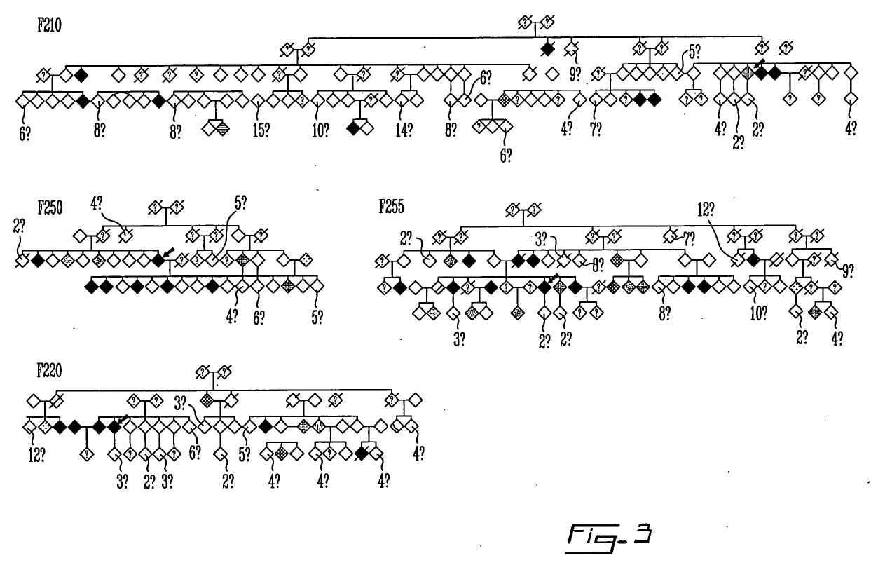

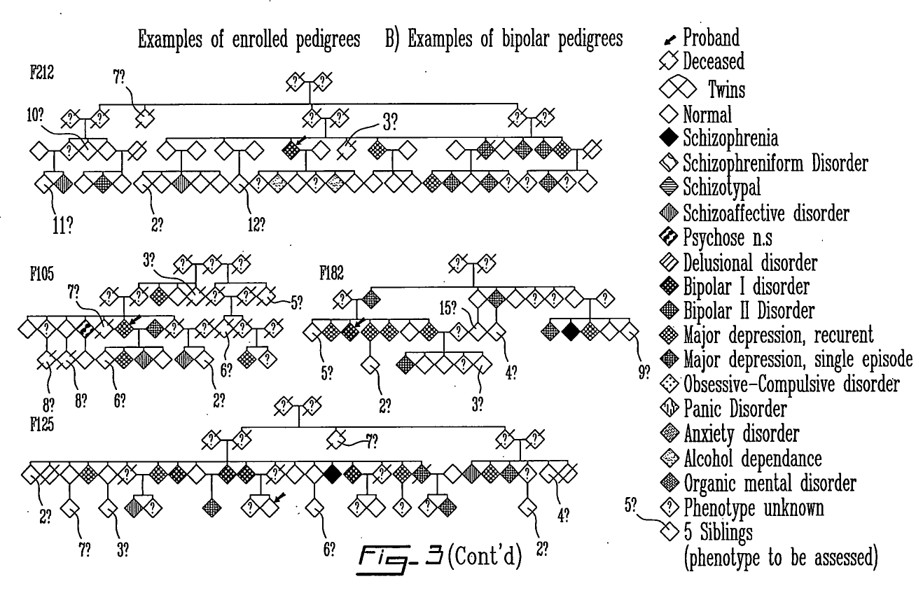

The sample consists of 46 multigenerational kindreds (N=980 family members and 97 unrelated controls to determine allele frequencies; sample 1=480 and sample 2=500). In all, 16 multigenerational pedigrees densely affected by BP (≦15% of SZ spectrum disorders in the pedigree) (FIG. 3 for examples), 15 affected by SZ (≦15% of BP spectrum disorder in the pedigree) (FIG. 3 for examples), and 15 mixed pedigrees affected almost equally (50±5%) by SZ and BP+spectrum disorders were considered. In sample 1, there are 468 genotyped and phenotyped individuals of whom N=134 were affected according to affected definition CLₙₐᵣ and 169 with affected definition CL_{bro}. We have 216 affected members (CL_{bro}) in sample 2. Homogeneous pedigrees were given a priority for enrolment, but we found that around 30% of the enrolled kindred's ended up as mixed after the blind diagnoses, after being formerly assessed as homogeneous at unblind BED (Best-Estimate Diagnosis).

Best-Estimate Diagnostic (BED) Procedure

Initially, all subjects were personally informed about the investigation and a signed consent was obtained. This BED is based on multiple sources of information, i.e., an audio-taped SCID interview, all available medical records and information gathered from several family respondents. 96% of the subjects have medical records and 86% have been directly interviewed. All this information was collected blind to genotypes by experienced and trained research assistants (psychiatric nurses). In a first step, under the supervision of research psychiatrists, a professional assistant highlights, from the medical records, all pertinent clinical information and summarizes it. She also summarizes the personal interview and family history interviews and writes out a differential diagnosis while rating the presence or absence of each diagnostic criterion according to DSM-IV, DSM-III-R, and RDC. After all available information has been gathered, a consensus diagnosis for each episode and a consensus lifetime diagnosis are made by the field investigators. In a second step, a diagnosis and a certainty level (definite, probable, possible) on diagnosis are done by a panel of 4 psychiatrists kept blind to genetic markers, predominant pedigree diagnosis category (SZ), family relationships, the field diagnosis (made in the previous step), and clinician's prior diagnoses. The board members were kept blind by providing them with edited raw clinical information including the audiotaped SCID, medical records and information from relatives.

### EXAMPLE III

### The Molecular Methods Used in This Study

All the methodologies required for cell immortalization and genotyping s are well established according to Maziade et al., (Maziade et al., 2001b, Mol. Psy. 6(6) 684-93). The cell immortalization success rate is over 95%, and the lymphoblastoid bank for SZstudies has around 1,200 cell lines (N=980 family members of SZ and BP kindred's; N=97 normal controls; N=115 unrelated SZ pro-bands).

Genotyping: Microsatellites

The DNA polymorphisms used are highly informative di-, tri-, and tetranucleotide microsatellite repeats for which PCR primers are synthesized (Alpha DNA, Montreal) after adding a M13 tail to the forward primer. A semi-automated high-throughput genotyping procedure using laser infrared automatic DNA sequencers, and automated genotyping software (SAGA) from LICO™ was used. The PCR amplification of microsatellites is routinely performed and well known in the art. For microsatellites and SNPs, genotypes are called automatically using the software SAGA (LICOR). After automatic genotyping, which is read blind to the phenotypes, manual editing of the results is performed, if needed. Results are then stored in a local database where Mendelian inheritance is checked using the computer software PedCheck (O'Connell & Weeks, 1998, Am J. Hum. Genet. 63(1) 259-66). Subjects who failed the Mendelian test are reanalyzed completely, i.e., from the PCR to the genotyping.

### EXAMPLE IV

### The Statistical Analysis Used in this Study

Detecting Genes Using Linkage and Association Studies

Association analyses serve as a method for fine mapping once an approximate location for a disease gene has been found by linkage analysis. This strategy was among the statistical approaches to gene mapping discussed by and is in agreement with position that a joint linkage and association analysis may have greater efficiency than either method considered alone. The details of each of these two complementary approaches are provided below.

Linkage Analysis

Model-based (or parametric) linkage analyses were used as the primary statistical method given growing evidence that they are more powerful than model-free (or non-parametric) analyses (Abreu et al., 1999, Am J. Hum. Genet. 65(3) 847-57; Durner et al., 1999, Am J. Hum. Genet. 64(1) 281-9) even when the mode of inheritance is unknown, provided that at least 1 dominant and one recessive model are considered. One dominant and one recessive model with disease gene penetrance values shown in Table 4 was defined.

TABLE 4
Penetrance parameter values^{a}
MODE OF INHERITANCE

| AFFECTION | AGE | RECESSIVE | | | DOMINANT | | |
|---|---|---|---|---|---|---|---|
| STATUS | CLASS | dd | Dd | DD | dd | Dd | DD |
| AFFECTED | ≦19 | 0.00025 | 0.00025 | 0.22 | 0.00012 | 0.22 | 0.22 |
| | 20-24 | 0.00025 | 0.00025 | 0.22 | 0.00012 | 0.22 | 0.22 |
| | 25-29 | 0.00014 | 0.00014 | 0.12 | 0.00006 | 0.12 | 0.12 |
| | 30-34 | 0.00009 | 0.00009 | 0.08 | 0.00004 | 0.08 | 0.08 |
| | ≧35 | 0.00008 | 0.00008 | 0.07 | 0.00004 | 0.07 | 0.07 |
| UNAFFECTED | ≦19 | 0.00025 | 0.00025 | 0.22 | 0.00012 | 0.22 | 0.22 |
| | 20-24 | 0.00050 | 0.00050 | 0.43 | 0.00023 | 0.43 | 0.43 |
| | 25-29 | 0.00063 | 0.00063 | 0.55 | 0.00029 | 0.55 | 0.55 |
| | 30-34 | 0.00072 | 0.00072 | 0.63 | 0.00033 | 0.63 | 0.63 |
| | ≧35 | 0.00080 | 0.00080 | 0.70 | 0.00037 | 0.70 | 0.70 |
| PROBABLY | — | 0.00016 | 0.00016 | 0.07 | 0.00007 | 0.07 | 0.07 |
| AFFECTED^{b} | | | | | | | |
| POSSIBLY | — | 0.00100 | 0.00100 | 0.07 | 0.00070 | 0.07 | 0.07 |
| AFFECTED^{b} | | | | | | | |

^{a}The penetrance values refer to the probability of having a given affection status conditional on the stage class and each of the three possible genotypes at the disease locus, i.e. dd, Dd and DD where the allele D identifies the susceptibility allele (for unaffected subjects, the LINKAGE program will use one minus these values as the probability of being unaffected in a given class age).
^{b}The liability class assigned to subjects with a probable or possible affection status was not age-dependent and took into account the certainty of diagnosis by increasing the phenocopy rate, as suggested by Ott (1989). The disease gene frequency used for the recessive and dominant model was respectively 0.10 and 0.001.

Both models specify age-dependent penetrances and take into account the certainty of diagnosis by increasing the phenocopy rate in liability classes corresponding to probable and possible diagnoses (last two lines in Table 4), as suggested by Ott (Ott J. 1989, Proc. Natl. Acad. Sci USA. 86(11) 4175-8). Two-point and three-point lod scores were computed using the FASTLINK version of the LINKAGE programs (Schaffer, A A. 1996, Hum. Hered. 46(4) 226-35). FASTLINK can deal properly with loops (Terwilliger & Ott, 1994 Compléter SVP Terwilliger J D, Ott J. Handbook of human genetic linkage. Johns Hopkins University Press, Baltimore). The analyses are first carried out using both affected and unaffected subjects, and then using affected-only to reduce the impact of a misspecification in the penetrance parameter.

For each marker, a mod score was obtained by maximizing the lod score over the eight possible combinations resulting from using two affected statuses two models of transmission (dominant vs. recessive) and two types of analyses (affected-unaffected vs. affected-only). Although using a mod score approach yields greater power to detect linkage than using a single model, it will inevitably inflate the rate of type I error. Hence, correction was applied for multiple testing by raising thresholds for genome-wide significance level by 0.70, following the guidelines of Hodge et al. (Hodge SE, et al., Am. J. Hum. Genet. 60 (1) 217-27). Therefore, the stringent adjusted Z criteria for assessing the significance level of the results are 4.0 for significant linkage, 2.6 for suggestive linkage, and 1.9 for confirmatory linkage in regions where significant linkage was previously reported.

Complex Modeling

Epistatic effects were searched in the family sample using a three-step strategy. First, single-locus (SL) analyses was performed, which consists of analysing each marker (representing a particular locus on a chromosome) at a time. Second, a thorough investigation of correlations between family lod scores resulting from the SL analyses was done. Under epistasis (i.e. at least 2 genes interacting), the SL lod scores to be positively correlated among pedigrees were targeted, while under independent action the SL lod scores should be negatively correlated. Third, once a positive correlation had been detected, epistasis was assessed using a two-locus lod score. In such two-locus analysis, the probability of linkage at two interacting loci is estimated simultaneously and a global index of the evidence epistasis is thus obtained.

### EXAMPLE V

### Characterization of the Complex SZ Phenotypes

Phenotype issues are now largely recognized in the art to which it pertains as a major issue in psychiatric genetics

Characterization of the Syndromal Phenotype and Effect of Blindness

Diagnostic accuracy is a basic prerequisite to successful linkage analyses given the sensitivity of these analyses to diagnostic misclassification. Hence, we have set up a very stringent best-estimate diagnostic (BED) method in two steps:

first, a diagnosis made by the field team who was unblind to the predominant diagnosis of the pedigree (i.e., SZ vs. BP). Second, a diagnosis was performed by a board of 4 research psychiatrists who were blind to the primary diagnosis of the pedigree. The blindness of the latter diagnosis was made possible by the studying concurrently SZ and BP pedigrees, since this cannot be achieved when a research group is studying only one disorder at a time, as is usually done. An important diagnostic bias, i.e., that in case of disagreement between the blind and the unblind BED, the unblind BED was in greater continuity with the most prevalent diagnosis in the pedigree. Thus, the blind BED method allowed us to avoid such a bias and therefore probably reduced the risk for false-positive diagnoses. The stringency of the BED method may be an explanation for the strength of the linkage results in sample 1. Moreover, the using blind BED may explain why a few of the pedigrees who were considered relatively “pure” by the field team (i.e., affected almost exclusively by disorders included either in the SZ or the BP spectrum) turned up to be mixed (i.e. affected almost equally by disorders from both spectrums) after the blind diagnoses.

Characterization of Dimensional Phenotypes

Whereas National Institute for Health (NIMH) and other agencies recently called for a fresh start with the collection of well-characterized families with extended-phenotypic measurements, in addition to DSM classifications, These costly measurements were already gathered since 1991 (Maziade et al., 1995b, Am J. Psychiatry 152(10) 1458-63). A factor analysis of the 8 SANS and SAPS psychotic dimensions was reported in the familial sample that was among the first to demonstrate that the Liddle's 3-factor model of psychotic symptom dimensions (negative, psychoticism, disorganized) was common to SZ and BP. In a furher factor analysis, the CASH manic and depressive dimensions were added to the psychotic dimensions: we observed that the positive and negative dichotomy remained but that a third factor comprising the affective dimensions was present (see Table 5).

TABLE 5
Factor Analysis^{a} at the Dimensions Level in the Total Sample (N=223) of
DSM-III-R Schizophrenic (N₁=118) and Bipolar (N₂=105) Patients During the
Acute and the Stabilized Phases

| | Acute phase | | | Stabilized phase | | |
|---|---|---|---|---|---|---|
| Symptom | Negative | Positive | Affective | Negative | Positive | Affective |
| Anhedonia | 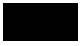 | 0.30 | −0.03 | 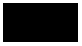 | 0.33 | 0.02 |
| Apathy | 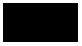 | 0.33 | 0.01 | 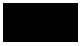 | 0.28 | 0.08 |
| Affective blunting | 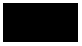 | 0.17 | −0.17 | 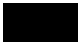 | 0.14 | −0.08 |
| Alogia | 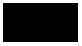 | 0.35 | −0.12 | 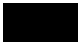 | 0.27 | 0.08 |
| Delusions | 0.16 | 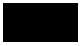 | −0.25 | 0.20 | 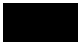 | 0.08 |
| Hallucinations | 0.35 | 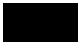 | −0.34 | 0.23 | 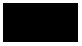 | —0.12 |
| Bizarre behavior | 0.30 | 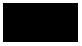 | 0.02 | 0.48 | 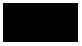 | 0.16 |
| Thought disorder | 0.15 | 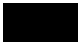 | 0.13 | 0.26 | 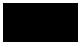 | 0.23 |
| Mania | 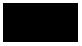 | 0.13 | 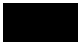 | −0.20 | 0.16 | 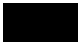 |
| Depression | −0.04 | −0.14 | 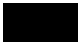 | 0.24 | 0.00 | 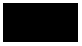 |
| Eigenvalue | 3.17 | 2.65 | 1.38 | 3.25 | 2.75 | 1.55 |
| % of variance | 32 | 27 | 14 | 33 | 28 | 16 |

^{a}Principal Component Analysis was done with Varimax rotation

Thirdly, the factor analysis was performed directly on the CASH psychotic and affective symptoms, instead of on the “a priori” categories of symptoms. A stable structure in the familial sample in acute phases and stabilized intervals of illness made up of 4 factors (negative, Schneiderian, manic, depressive), confirming again the presence of the positive/negative dichotomy in addition to affective dimensions. A fifth orthogonal factor in the stabilized phase only was observed (Table 6): a thought disorder factor. A number of recent factor analyses of symptoms in major psychoses show a surprising degree of consistency of these 5 factors across samples and instruments.

As a another strategy, we used the dimensional phenotypes to define the phenotypes that fit best the significant linkages obtained in the primary strategy using the syndrome phenotype (i.e., DSM diagnoses).

TABLE 6
Factor Analysis of the 82^{a} Psychotic and Affective Symptoms in Sample 1^{b}
in Lifetime Stabilized intervals

| Symptom | Factor 1 | Factor 2 | Factor 3 | Factor 4 | Factor 5 | |
|---|---|---|---|---|---|---|
| Psychotic symptoms | | | | | | |
| Delusions of reference | 0.23 | 0.20 | 0.15 | 0.47 | 0.20 | |
| Persecutory delusions | 0.17 | 0.23 | 0.26 | 0.39 | 0.35 | |
| Grandiose delusions | 0.14 | 0.45 | 0.43 | 0.53 | −0.10 | |
| Somatic delusions | 0.15 | 0.38 | 0.15 | 0.34 | 0.30 | |
| Delusions of sin or guilt | 0.03 | −0.02 | −0.04 | 0.34 | 0.32 | |
| Delusions of jealousy | 0.08 | 0.02 | −0.04 | −0.10 | 0.22 | |
| Delusions of nihilism | 0.04 | 0.86 | 0.28 | −0.09 | −0.03 | |
| Religious delusions | 0.28 | 0.24 | 0.33 | 0.43 | 0.00 | |
| Delusions of mind reading | −0.01 | 0.55 | 0.31 | 0.29 | 0.12 | |
| Thought insertion | 0.03 | 0.80 | 0.12 | 0.06 | 0.03 | |
| Thought withdrawal | 0.05 | 0.85 | 0.24 | −0.04 | −0.04 | |
| Systematized delusions | 0.42 | 0.48 | 0.00 | 0.14 | 0.07 | |
| Bizarre or fantastic delusions | 0.20 | 0.54 | −0.03 | 0.41 | 0.05 | |
| Hallucinations | | | | | | |
| Auditory hallucinations (voices, | 0.18 | 0.45 | −0.06 | 0.41 | 0.40 | |
| noises, music) | | | | | | |
| Auditory hallucinations (voices | 0.22 | 0.57 | 0.03 | 0.14 | 0.36 | |
| commenting) | | | | | | |
| Visual hallucinations | 0.02 | 0.64 | 0.01 | 0.49 | 0.12 | |
| Somatic or tactile hallucinations | 0.09 | 0.72 | 0.05 | 0.19 | 0.12 | |
| Olfactory or gustatory | 0.10 | 0.66 | 0.05 | 0.34 | −0.04 | |
| hallucinations | | | | | | |
| Thought disorders | | | | | | |
| Incoherence (word salad, | 0.37 | 0.21 | 0.20 | 0.73 | −0.08 | |
| schizophasia) | | | | | | |
| Derailment (loose associations) | 0.31 | 0.28 | 0.16 | 0.69 | −0.07 | |
| Tangentiality | 0.13 | 0.06 | −0.04 | 0.65 | 0.11 | |
| Illogicality | 0.37 | 0.11 | 0.03 | 0.77 | −0.08 | |
| Circumstantiality | 0.02 | 0.26 | 0.11 | 0.17 | 0.04 | |
| Pressure of speech | 0.02 | 0.14 | 0.61 | 0.42 | 0.11 | |
| Clanging | 0.12 | 0.49 | 0.31 | −0.02 | −0.06 | |
| Bizarre behaviors | | | | | | |
| Clothing and appearance | 0.33 | 0.61 | 0.01 | 0.24 | 0.00 | |
| Social and sexual behavior | 0.31 | 0.35 | 0.19 | 0.54 | 0.04 | |
| Aggressive and agitated behavior | 0.08 | 0.26 | 0.18 | 0.47 | 0.34 | |
| Ritualistic or stereotyped | 0.39 | 0.09 | 0.11 | 0.00 | 0.03 | |
| behavior | | | | | | |
| Anhedonia | | | | | | |
| Recreational interests and | 0.44 | 0.05 | 0.14 | 0.25 | 0.70 | |
| activities | | | | | | |
| Ability to feel intimacy and | 0.48 | 0.19 | −0.09 | 0.25 | 0.49 | |
| closeness | | | | | | |
| Relationships with friends and | 0.57 | 0.20 | −0.09 | 0.29 | 0.46 | |
| peers | | | | | | |
| Avolution - apathy | | | | | | |
| Grooming and hygiene | 0.54 | 0.29 | 0.12 | 0.30 | 0.19 | |
| Physical anergia | 0.54 | 0.13 | 0.08 | 0.03 | 0.62 | |
| Affective flattening | | | | | | |
| Flat affect | 0.77 | −0.06 | −0.01 | 0.07 | 0.26 | |
| Unchanging facial expression | 0.83 | 0.01 | −0.02 | 0.21 | 0.18 | |
| Decreased spontaneous | 0.73 | 0.03 | 0.05 | −0.04 | 0.06 | |
| movements | | | | | | |
| Paucity of expressive gestures | 0.63 | −0.03 | −0.09 | 0.16 | 0.08 | |
| Poor eye contact | 0.72 | 0.11 | −0.10 | −0.01 | 0.08 | |
| Affective nonresponsivity | 0.67 | −0.06 | −0.05 | 0.29 | 0.12 | |
| Alogia | | | | | | |
| Poverty of speech | 0.78 | 0.09 | −0.02 | 0.20 | 0.07 | |
| Blocking | 0.57 | 0.09 | 0.18 | 0.16 | −0.13 | |
| Increased latency of response | 0.59 | 0.09 | −0.04 | 0.15 | −0.06 | |
| Perseveration | 0.37 | 0.17 | 0.05 | 0.05 | 0.08 | |
| Grossly inappropriate affect | 0.67 | 0.16 | 0.11 | 0.40 | −0.02 | |
| Emotional turmoil | 0.28 | 0.26 | 0.28 | 0.22 | −0.11 | |
| Social inattentiveness | 0.60 | 0.38 | 0.01 | 0.08 | 0.12 | |
| Manic symptoms | | | | | | |
| Inflated self-esteem | 0.04 | 0.26 | 0.78 | 0.14 | −0.10 | |
| Decreased need for sleep | −0.02 | 0.20 | 0.72 | 0.04 | 0.09 | |
| Increased talkativeness | −0.08 | 0.08 | 0.79 | 0.21 | 0.18 | |
| Flight of ideas | 0.06 | 0.24 | 0.77 | 0.01 | −0.09 | |
| Distractibility | 0.19 | 0.32 | 0.63 | −0.14 | 0.03 | |
| Increase of activity | −0.02 | 0.08 | 0.80 | 0.02 | 0.18 | |
| Poor judgment | −0.15 | −0.13 | 0.73 | 0.11 | 0.05 | |
| Euphoric mood | 0.04 | 0.03 | 0.73 | 0.16 | 0.32 | |
| Depressive symptoms | | | | | | |
| Dysphoric mood | −0.06 | −0.11 | 0.39 | 0.02 | 0.59 | |
| Loss of interest or pleasure | 0.07 | −0.05 | 0.21 | −0.04 | 0.81 | |
| Diminished ability to think or | 0.14 | 0.08 | 0.47 | −0.07 | 0.40 | |
| concentrate | | | | | | |
| Recurrent thoughts of | −0.08 | 0.12 | 0.23 | 0.21 | 0.49 | |
| death/suicide | | | | | | |
| Eigenvalue | 8.22 | 7.17 | 6.41 | 5.69 | 4.08 | Total = 31.6 |
| % of variance | 13.90 | 12.20 | 10.90 | 9.60 | 6.90 | Total = 53.5% |

^{a}Among the 82 items, 23 were excluded due to a very low rate of responses or to an insufficient reliability of the item.
^{b}The factor analysis is based on the 161 subjects among sample 1 who had no missing values on each of the 82 symptoms.

While the invention has been described in connection with specific embodiments thereof, it will be understood that it is capable of further modifications and this application is intended to cover any variations, uses, or adaptations of the invention followings in general, the principles of the invention and including such departures from the present disclosure as come within known or customary practice within the art to which the invention pertains and as may be applied to the essential features hereinbefore set forth, and as follows in the scope of the appended claims.

1. A method for determining susceptibility of an individual to develop at least one of SZ or associated clusters of symptoms thereof before the illness appears comprising;
a) characterizing in an individual a genotype markers combination of at least one genotype markers located in genotype region 6p22.3 and at least one genotype marker located in genotype region 18q2 1.1; and
b) comparing genotype markers of said individual of step a) with genotype markers of a patient diagnosed for SZ or associated clusters of symptoms thereof located in genotype regions 6p22.3 and 18q21.1, wherein detecting presence of genotype markers of said patient diagnosed for SZ or associated clusters of symptoms thereof in genotype regions 6p22.3 and 18q21.1 of said individual is representative of the susceptibility of to develop at least one of SZ or associated clusters of symptoms thereof.

2. The method of claim 1, wherein level of correlation of said genotype markers of said individual with genotype markers of said patient diagnosed for SZ or associated clusters of symptoms thereof is representative of the level of the susceptibility to develop SZ or associated clusters of symptoms thereof.

3. The method of claim 1, wherein the genotype markers combination is an epistatic combination.

4. The method of claim 1, wherein frequency of genotype markers of said individual is about 20 to 100% equivalent to the frequency of genotype markers of a patient diagnosed for SZ or associated clusters of symptoms thereof.

5. The method of claim 1, wherein said characterizing allows to define subtype of SZ.

6. The method of claim 1, wherein said SZ is a schizophreniform disorder, an achizotypal personality disorder, psychosis, or a schizoaffective disorder.

7. The method of claim 1, wherein said characterizing is determining DNA sequence, RFLP, hybridization pattern, or gel migration pattern of said genotype markers.

8. The method of claim 1, wherein said genotype markers combination includes combination of at least one marker between loci D18S65 and D18S64 on chromosome 18 with at least one marker between loci D6S1267 and D6S89 on chromosome 6.

9. A method for diagnosing illness or predicting severity of illness of at least one of SZ or associated clusters of symptoms thereof of an individual comprising;
a). characterizing in an individual a genotype markers combination of at least one genotype markers located in genotype region 6p22.3 and at least one genotype marker located in genotype region 18q21.1; and
b) comparing genotype markers of said individual of step a) with genotype markers of a patient diagnosed for SZ or associated clusters of symptoms thereof located in genotype regions 6p22.3 and 18q21.1, wherein detecting presence of genotype markers of said patient diagnosed for SZ or associated clusters of symptoms thereof in genotype regions 6p22.3 and 18q21.1 of said individual is representative of the presence of illness or the severity of illness of at least one of SZ or associated clusters of symptoms thereof of said patient.

10. The method of claim 9, wherein said diagnosing or predicting is performed before or after symptoms of at least one of SZ or associated clusters of symptoms thereof occurs.

11. The method of claim 9, wherein the level of correlation of said genotype markers of said individual with genotype markers of said patient diagnosed for SZ or associated clusters of symptoms thereof is representative of the level of the illness or severity of illness.

12. The method of claim 9, wherein the genotype markers combination is an epistatic combination.

13. The method of claim 9, wherein frequency of genotype markers of said individual is about 20 to 100% equivalent to the frequency of genotype markers of a patient diagnosed for SZ or associated clusters of symptoms thereof.

14. The method of claim 9, wherein said characterizing allows to define subtype of SZ.

15. The method of claim 1, wherein said SZ is a schizophreniform disorder, an achizotypal personality disorder, psychosis, or a schizoaffective disorder.

16. The method of claim 9, wherein said characterizing is determining DNA sequence, RFLP, hybridization pattern, or gel migration pattern of said genotype markers.

17. The method of claim 9, wherein said genotype markers combination includes combination of at least one marker between loci D18S65 and D18S64 on chromosome 18 with at least one marker between loci D6S1267 and D6S89 on chromosome 6.
